(19) United States
(12) Patent Application Publication
(10) Pub. No.: US 2007/0179358 A1
(43) Pub. Date: Aug. 2, 2007

(54) Device for increased compliance with diabetes monitoring tests

(76) Inventor: Anita Greggs Perez, Chandler, AZ (US)

Correspondence Address: ETHERTON LAW GROUP, LLC, 5555 E. VAN BUREN STREET, SUITE 100, PHOENIX, AZ, 85008, US

(21) Appl. No.: 11/639,927

(22) Filed: Dec. 15, 2006

Related U.S. Application Data
(60) Provisional application No. 60/750,880, filed on Dec. 16, 2005.

(51) Int. Cl. A61B 5/00; G01N 33/487

(52) U.S. Cl. 600/300; 600/365; 204/403.01

(57) ABSTRACT

A device for increasing the compliance of a patient to a diabetes monitoring protocol. The device comprises a glucose measurement device and an entertainment device. Any glucose measurement device can be used, such as a test-strip reader, a breath analyzer, or a spectrometer. The entertainment device is any audio-visual playback device. In the preferred embodiment, the glucose measurement device is a test strip reader that is integral with an MP3 player.

### CROSS-REFERENCE TO RELATED APPLICATIONS

This application claims the benefit of co-pending U.S. Provisional Application No. 60/750,880 filed Dec. 16, 2005.

### FIELD OF INVENTION

This invention relates generally to diagnostic testing of a human body. More particularly, this invention relates to a diagnostic device having features that increase compliance with a glucose monitoring protocol.

### BACKGROUND

Diabetes is a disease in which the body does not produce or properly use insulin. Insulin is a hormone that is needed to convert sugar, starches and other food into glucose, which is the fuel cells need for on-going activity. Published studies indicate that at least 7% of the US population has diabetes, and about 70% of those with diabetes have been diagnosed. About 5-10% of all diabetics have Type I diabetes in which the pancreatic cells that produce insulin have been destroyed. Thus, there are no cells to produce the chemical that produces insulin. Type I diabetes is treated by supplying insulin by injection or pump. The balance of those with diabetes have Type II diabetes in which pancreatic cells produce insulin, but other cells in the body do not use insulin well to convert food into glucose. Type II diabetes is treated by diet, exercise, oral medications, insulin, or a combination thereof.

While diabetes cannot yet be cured, it can be controlled. If it is not controlled, complications result. For example, adults with diabetes have heart disease rates about 2 to 4 times higher than adults without diabetes. The risk for stroke is 2 to 4 times higher among people with diabetes. Diabetes is the leading cause of new cases of blindness among adults aged 20-74 and the leading cause of kidney failure. About 60-70% of people with diabetes have mild to severe forms of nervous system damage. The result of such damage includes impaired sensation or pain in the feet or hands, which may eventually result in amputation of the limb. In addition, people with diabetes are more susceptible to many other illnesses and, once they acquire these illnesses, often have worse prognoses than non-diabetics.

To control diabetes, it is necessary to monitor the level of glucose in the blood. The frequency of measurement varies from patient to patient, depending on a number of factors including the severity of the disease, type of diabetes, level of physical activity, eating habits, and other health issues. It is often necessary to determine the glucose level in blood several times a day. Consistently taking readings help patients manage their glucose levels better, thereby improving insulin and other therapies and helping to prevent complications.

A common method of blood glucose self-monitoring is to prick a finger or other area to release capillary blood, absorb a minute amount of blood onto a test strip, and insert the test strip into a monitor to measure the amount of glucose in the blood. Although not as common yet, the patent literature is replete with less invasive methods of determining glucose concentration in the blood, by direct measurement of glucose or indirect measurement of another substance or parameter that correlates to the amount of glucose in the blood. These include chemical or spectroscopic (transcutaneous infrared, fluorescence lifetime, pulsed laser photoacoustic, and far infrared) measurement of interstitial fluid from the skin obtained by reverse iontophoretic, electroosmotic, or thermal microporation sampling. Other approaches to glucose measurement are analysis of glucose in breath, sweat or tears; temperature of the blood; optical measurements of the aqueous humor of the eye including polarimetry, and radio wave impedance of skin. Devices for measuring glucose in the blood, whether directly or indirectly, are referred to in the art as blood glucose meters.

Portable glucose meters have been developed to help people check their blood sugar at home, school, work, and play. Each of the meters has a different set of features, and some have wired or wireless capabilities to allow the user to download the blood glucose readings to a health care provider which, herein, broadly includes physicians, nurses, diabetic counselors, disease management organizations and insurance providers. Other meters have data ports that allow wired download to computer. However, no standard has yet been created for the data download, resulting in frequent incompatibilities between the meter ports, cable terminals, and computer ports. It would be desirable to have a common data port for easier downloading of meter readings. Increased downloading would have the added benefit of enabling health care providers to provide expert interpretation of the readings, thereby better managing patient insulin and medication therapies and helping to prevent complications. Because an expert's reading of the data may decrease health costs by eliminating complications, a case can be made for insurance providers to begin paying health care providers to read and interpret the data, much like they are paid to interpret other test results. This, in turn, would also help increase compliance because the patient would be reminded by the health care provider to make glucose measurements.

Despite the improvement in meter portability and ease of use, compliance with the monitoring protocol continues to be a problem, especially for young people. Children and teens, ages 7-17, have poor compliance, which is not particularly surprising. However, more surprising is that the age group having the worst compliance is the 17-27 year-olds. Even though the failure to test may be life-threatening in the long run, most people do not recognize or acknowledge the long-term risk. There are several factors in non-compliance. For example, a portable glucose meter is an additional piece of equipment that the person needs to carry, so it gets left behind because it is forgotten until it is needed or it doesn't fit in a pocket or purse. Another problem is that a glucose meter is not commonly carried by most people, and being similar to peers is of primary importance in younger age groups, and of some significant importance to many others who do not want to broadcast their disease. It would be desirable to increase compliance with diabetes monitoring protocols by making it more convenient and less obvious to carry a monitor.

Recently, portable music and video players have become ubiquitous with the very same groups that have poor compliance with glucose monitoring. Owners of these audio/video devices derive great pleasure from them, because the devices are loaded with various forms of entertainment that the owner has selected. Users therefore carry them wherever they go. It would be desirable to provide a glucose meter that is integrated with a device that a person usually carries. It would be particularly desirable to provide a glucose meter that is integrated with a device that provides a person enjoyment.

There is a need to provide a device that increases testing compliance. Therefore, an object of this invention is to provide a testing device that is integral with an object that is commonly carried. Another object of this invention is to provide a testing device and entertainment. Another object is to provide a hand-held system that combines a testing device and an audio/video recorder player. A further object of this invention is to provide a testing device that is disguised as another device that a person regularly carries. Another object is to provide a testing device that has a universal download capability.

### SUMMARY OF THE INVENTION

The present invention is a device for increasing the compliance of a patient to a diabetes monitoring protocol. The device comprises a blood glucose meter and an entertainment device. Any blood glucose meter can be used, such as an electrochemical test-strip reader, a breath analyzer, or a transcutaneous spectrometer. The entertainment device is any audio/video device. In the preferred embodiment, the blood glucose meter is a test strip reader that is integral with an MP3 player that contains one or more audio or visual files selected by the patient.

### BRIEF DESCRIPTION OF THE DRAWINGS

FIG. 1 is a schematic of the preferred embodiment of the present invention

FIG. 2 is a perspective view of the exterior of the preferred embodiment of the present invention.

### DETAILED DESCRIPTION OF THE INVENTION

The present invention, generally referred to as device 10, combines a bio-sensor device for measuring biological data of the patient as part of a disease monitoring protocol and a portable entertainment device. In the preferred embodiment, the biological data being monitored is blood glucose levels as a part of controlling diabetes, however any other biological data for controlling other diseases can be measured, too. Other disease monitoring protocols include those to monitor asthma; chronic obstructive pulmonary disease including emphysema and chronic bronchitis; alcoholism or other diseases of addiction; high blood pressure and other cardiovascular problems; and weight loss or gain. Other biological data to be measured include nitric oxide, carbon dioxide, hematocrit, ketones, weight, pulse, blood pressure, blood cholesterol, and blood hormone levels.

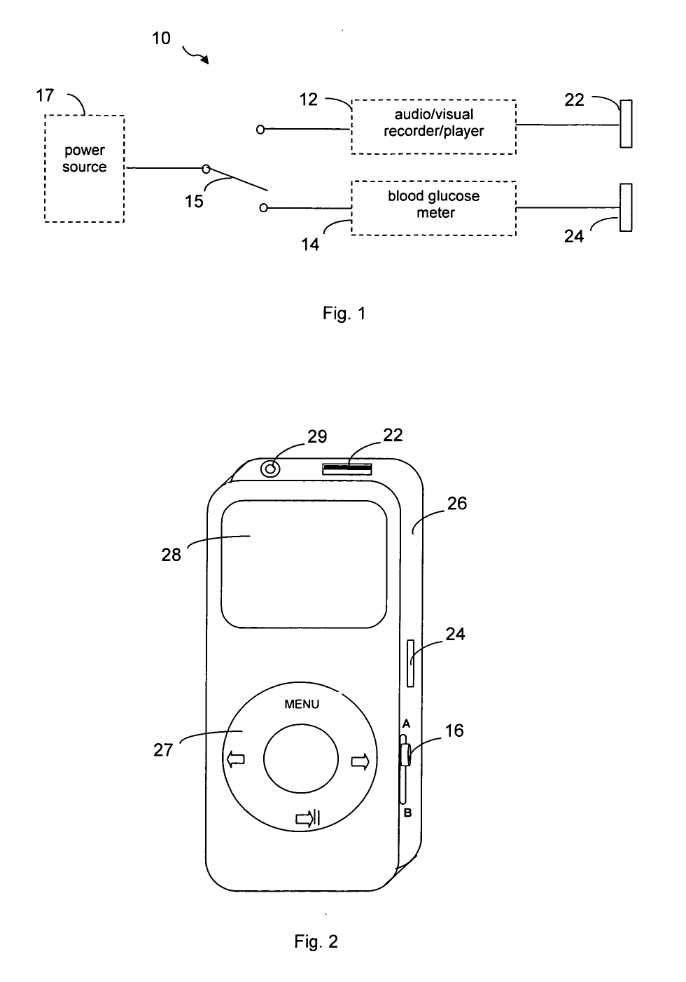

FIG. 1 shows a schematic illustration of the preferred embodiment. A blood glucose meter 14 is combined with an audio/visual player/recorder 12. Blood glucose meters are known in the art, and encompass a wide array of methods for collecting and analyzing the blood or other bodily substance to be tested. Some meters measure the glucose level directly; others measure indirectly by measuring a parameter in another substance that correlates to the amount of glucose in the blood. Substances measured that correlate to blood glucose levels include oxygen concentration, fructosamine and other glycated proteins, and hemoglobin A_{1c}.

A typical direct electrochemical glucose meter involves obtaining a small sample of interstitial fluid by creating a micropore. Typically the sample site is the fingertip, but can also be an alternate site such as the palmar region of the hand, base of thumb, forearm, upper arm, head, earlobe, chest, torso, abdominal region, thigh, calf, and foot including plantar region and toes. The sample of blood is placed on a disposable test strip, typically an absorbent material like filter paper, and the strip is put in the meter. The test strips are coated with chemicals (such as glucose oxidase, dehydrogenase, or hexokinase) that combine with glucose in blood. The present device 10 has an aperture 24 for inserting a test strip. The meter measures how much glucose is present. Meters do this in different ways, such as measuring the amount of electricity that can pass through the sample, the wavelength of light reflected from the test strip, or how much light reflects from it.

A diabetic patient with low blood sugar can have high levels of acetone on his breath. The biosensor in the device 10 may alternatively include a glucose meter with infrared spectrometry to measure levels of acetone in the breath. Similarly, the device may include a breath test for other ketones, as well as alcohol or other compounds containing the methyl group. Recent evidence has shown that nitric oxide levels are increased in the breath of people with asthma and that changes in nitric oxide levels may indicate whether or not treatment for asthma is working. Therefore, for monitoring asthma, the biosensor can be a breath test for nitric oxide.

Other non-invasive technologies for glucose testing that avoid drawing blood can also be used in which the sample analyzed is a portion of the human body in-situ, not a biological sample acquired from the human body. These include chemical or spectroscopic (transcutaneous infrared, fluorescence lifetime, pulsed laser photoacoustic, and far infrared) measurement of interstitial fluid from the skin obtained by reverse iontophoretic, electroosmotic, or thermal microporation sampling or bioimpedance. In bioimpedance, a small electric current is conducted through the skin and the impedance of skin tissue is measured. The measurement is correlated with directly-determined glucose levels to determine the glucose level from the newly measured impedance. Other approaches to glucose measurement are analysis of glucose in breath, sweat or tears; temperature of the blood; optical measurements of the aqueous humor of the eye, polarimetry, and radio wave impedance. Still other methods of determining blood glucose concentration include using other body fluids such as urine, tears, saliva or sweat.

The preferred meter 14 is a glucose test strip reader that provides for 20,000 strip insertions and 500 calibration strip insertions. The meter instructions and date/time stamp are offered in multiple languages. The glucose measurements are provided in multiple units, for example milligrams per deciliter and millimoles for European market. The meter's minimum requirements meet or exceed LifeScan Flash product specifications, including a 5 second test, less than 1 uL blood, 250 measurement memory, test-averaging, downloadable results, and code-chip or test-strip calibration.

Some models may have automatic timing, error codes and signals, or barcode readers to help with calibration. Some meters may have a large display screen or spoken instructions for people with visual impairments. Some meters may have memory capability that can record and store a number of test results. Various models of the present invention will combine these features as desired for the target customer.

Portable audio/video player/recorders are known in the art. The preferred version of the portable entertainment device will be equipped with electronic circuity to play music, specifically MPEG audio layer-3 files, known in the art as an MP3 player. MP3 players, such as the Apple iPod®, are widely available commercially, as well as component kits to build them. In addition to playing sound files, the device may play video files. The device may play and record any audio or video formats including MP3 Pro, MP4 , MC, M, AiFF, ATRAC3 and ATRAC3plus, AVI, FLAC, OGG, WMA, WAV, AVI, MPEG, MOV, WMV, ASF, RM. 3GPP and 3GPP2.

To increase compliance, the entertainment device should contain entertainment content that is appealing to the patient. To that end, in the preferred embodiment, the audio and video content is selected primarily by the patient. The entertainment content can be downloaded by the patient to the entertainment device, as is known in the art, including by means of wired or wireless connection to a computer, to the internet, another entertainment device or other source, or over the air. Alternatively, the entertainment device can come pre-loaded with entertainment content that is appealing to the patient. The entertainment content can be audible, visible, or both.

The entertainment content preferably comprises music, but may also be narrative, such as a book read aloud, or even non-musical sounds such as soothing jungle or rain forest sounds. The entertainment may also comprise videos to be visually displayed on the screen 28, such as an educational video, a sports video, a crazy video, a humorous video, a pornographic video, a cartoon video, a still image, a writing, a painting, a work of art, a drawing, an informative table, a graphic, or other visual display other than a video.

The entertainment device is driven by an operating system. Preferably the operating system is compatible with Windows 98/2000/ME/XP or above and Apple's MAC OS 10 and above, and includes standard features such as drag and drop compatibility, the ability to download and manage songs and videos, and a user friendly menu system to aid navigation. The preferred version also has an equalizer mode with normal/bass/jazz/rock/classic and a repeat function for those songs the user can't get enough of. Preferably the device will have a speaker for emitting the audio content, and may also have an outlet 29 for stereo headphones. Alternative versions of the device may include a voice recorder, terrestrial or satellite radio tuner, cell phone, calendar or email receiver/transmitter, or a combination thereof.

One embodiment of the device will also allow the user to download and store music, images or other audio/video files. The device has an associated download aperture 22 for downloading content from a computer. Preferably the device connects with a computer using a USB port, preferably with at least USB 2.0 Compatibility. Alternatively, the device can connect with FireWire cable under the IEEE 1394-1995, IEEE 1394a-2000, and IEEE 1394b standards. Alternate versions may connect wirelessly. Preferably the device will read data at least 500 kbps and record at 400 kbps. In the preferred embodiment, the device 10 receives entertainment content, but cannot transmit entertainment content or anything else to other devices: it is disabled from doing so. This adds a layer of security and privacy of biomeasurements and medical data for the patient. An alternate embodiment enables the user to transfer and store music, images or other audio/video files.

The device may be capable of storing numerous entertainment files and glucose readings. The device preferably has a permanent or removable memory circuit capable of storing at least 256 MB and preferably 4 GB of data. The memory is preferably the same as that found in a conventional jump drive—also known as a USB drive, flash drive, keychain drive, or disk-on-key. The device preferably uses flash memory which is a type of constantly-powered nonvolatile memory that can be erased and reprogrammed. The jump drive does not require rebooting after it is attached, does not require batteries or an external power supply, and is not platform dependent. This makes it convenient for transferring data between the device 10 and another device such as a laptop or desktop computer or other entertainment device. The jump drive may have password protection.

The device 10 incorporates a selector switch 15 to switch from the MP3 operation to the glucose testing operation. In the preferred embodiment, the switch is a simple A/B switch 16 that physically moves the connection from the meter 14 to the audio/video player recorder 12. Alternatively, the switch 15 may comprise rubber membrane buttons, touchpads, or a pin switch to shift from blood glucose tests to entertainment functions. The switch 15 may be software driven and made by navigating the menu displayed on the screen 28. Alternate versions for switching from one mode to the other include touch screen, dials, up and down arrows, voice recognition, or other toggles.

The device 10 includes at least one power source 17. See FIG. 1. The device is preferably battery powered, with removable, rechargeable batteries. Alternatively, the device may use removable batteries that are not rechargeable; rechargeable batteries that are not removable; or batteries that are rechargeable by plugging the device into the computer with the USB connector.

The components are preferably combined in a single, hand-held unit 26 with a durable case that has keys or other input devices 27 for operating the audio/visual player/recorder 12 and meter 14, and a screen 28 for navigating a menu displayed on the screen. The glucose meter 14 may be physically attached to the entertainment device 14, or they may be integral, housed in a single case. The configuration will depend on a number of factors, including appearance, size, and need for keeping the bodily fluids and biosensor chemicals separated from the entertainment device. The overall device measurements may vary, depending on the model, but the device will be sized to be conveniently hand-held. General dimensions of the outer case are preferably about 8 cm×5 cm×2 cm, with a 3 cm×3 cm screen. The screen 28 is preferably an LCD screen with a backlight. The preferred device weighs 90-120 g (including batteries). See FIG. 2.

The device is able to withstand multiple drops during life and rough handling in general, like other audio/video recorder/players. The case is made of metal or plastic, and the choice of plastic must assume potential cleaning with disinfectants, including soap and water, alcohol or ammonia. The case may have a compartment to hold test strips or lancets. The design protects the screen 28 from cracking and maintains an electrical circuit between the battery 17 and audio/visual player/recorder 12 and blood glucose meter 14, which may be combined on a single circuit board. Further, the case is insulated to protect the electronics from electrostatic discharge of up to 15KV. The screen 28 is preferably scratch-resistant. The case will preferably have a label recess and rubber feet or similar mechanism for elevating the device above the surface it is resting on and a transparent cover over the screen.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments falling within the scope of the appended claims.

I claim:

1. A device for increasing compliance of a patient to a disease monitoring protocol, the device comprising:
a. a bio-sensor for measuring biological data of the patient as part of a disease monitoring protocol; and
b. a portable entertainment device.

2. The device of claim 1 wherein the bio-sensor is a blood glucose meter.

3. The device of claim 1 wherein the entertainment device comprises entertainment content selected by the patient.

4. The device of claim 3 wherein the entertainment content is audibly emitted from the device.

5. The device of claim 3 wherein the entertainment content is displayed visually on the device.

6. The device of claim 3 wherein the device cannot transmit entertainment content electronically to other devices.

7. The device of claim 1 wherein the device cannot transmit data measured by the bio-sensor to other devices.

8. The device of claim 1 wherein the entertainment device is a multimedia player.

9. The device of claim 1 wherein the entertainment device is an MP3 player.

10. The device of claim 1 wherein the entertainment device is a radio.

11. The device of claim 1 wherein the bio-sensor is disposed in the same housing as the entertainment device.

12. A device for increasing compliance of a patient to a blood glucose measurement protocol, the device comprising:
a. a blood glucose meter; and
b. a portable, hand-held device for playing music.

13. The device of claim 12 further comprising a memory circuit for storing music, wherein music that is selected by the patient is stored in the memory circuit.

14. The device of claim 12 wherein the music is audibly emitted from the device.

15. The device of claim 12 wherein the device cannot transmit music electronically to other devices.

16. The device of claim 12 wherein the device cannot transmit data measured by the blood glucose meter to other devices.

17. The device of claim 12 wherein the blood glucose meter is disposed in the same housing as the hand-held device for playing music.

18. The device of claim 12 wherein the blood glucose measurement device further comprises a spectrometer.

19. The device of claim 12 wherein the blood glucose measurement device further comprises an electrochemical test strip reader.

20. The device of claim 12 wherein the entertainment device is a radio.
